(19) United States
(12) Patent Application Publication
(10) Pub. No.: US 2007/0105032 A1
(43) Pub. Date: May 10, 2007

(54) Titanyl phthalocyanine crystal and manufacturing method thereof and electrophotographic photoreceptor and electrophotographic imaging apparatus using the photoreceptor

(75) Inventors: Saburo Yokota, Suwon-si (KR); Moto Makino, Suwon-si (KR); Hwan-koo Lee, Suwon-si (KR); Beom-jun Kim, Yongin-si (KR); Seung-ju Kim, Suwon-si (KR); Ji-Young Lee, Suwon-si (KR)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Samsung Electronics Co., Ltd.

(21) Appl. No.: 11/588,290

(22) Filed: Oct. 27, 2006

(30) Foreign Application Priority Data
Nov. 4, 2005 (KR) 10-2005-0105576

(51) Int. Cl. G03G 5/06; G03G 5/047; C07D 487/22

(52) U.S. Cl. 430/59.5; 430/78; 399/159; 540/141

(57) ABSTRACT

A titanyl phthalocyanine crystal has a major absorption peak at a wavelength of 780 nm±10 nm in the visible-infrared absorption spectrum and a minor absorption peak has an intensity of ¾ or less of the major absorption peak at 700 nm±10 nm. A method of manufacturing the crystal, an electrophotographic photoreceptor including the crystal as a charge generating material, and an electrophotographic image forming apparatus are also provided. The electrophotographic photoreceptor using the titanyl phthalocyanine crystal as the charge generating material has good sensitivity and a good residual current property and in addition, good stability.

### CROSS-REFERENCE TO RELATED PATENT APPLICATION

This application claims the benefit of Korean Patent Application No. 10-2005-0105576, filed on Nov. 4, 2005 in the Korean Intellectual Property Office, the disclosure of which is herein incorporated by reference in its entirety.

### BACKGROUND OF THE INVENTION

1. Field of the Invention

The present invention relates to a titanyl phthalocyanine crystal having a novel crystal type with a high charge generating efficiency. The invention is also directed to a method of producing the titanyl phthalocyanine crystal. The invention is further directed to an electrophotographic photoreceptor with a high sensitivity and an electrographic image forming apparatus using the titanyl phthalocyanine crystal.

2. Description of the Related Art

Phthalocyanine compounds show good photoconductivity to light in the range of visible light to near infrared rays, and thus, are widely used as a photoelectric material for a charge generating material of an electrophotographic photoreceptor or an organic solar battery. Titanyl phthalocyanine compounds having a tetravalent titanium atom bonded with a hydrogen and oxygen atom are used mostly for their good sensitivity and stability.

Like many other phthalocyanine compounds, titanyl phthalocyanine compounds have various crystal forms at a room temperature.

For example, U.S. Pat. No. 4,664,997 discloses a titanyl phthalocyanine crystal having a major absorption peak at a wavelength of around 760 nm. This crystal type is generally known as a β-type and is the most stabile and has the lowest sensitivity.

U.S. Pat. No. 4,728,592 discloses an α-type titanyl phthalocyanine having a major absorption peak at a wavelength of around 830 nm. The sensitivity of the α-type is 1.5 times greater than that of the β-type, which is effective for producing a high efficiency electrophotographic photoreceptor.

U.S. Pat. No. 4,898,799 discloses a crystal having a major peak at Bragg's 2θ angle of about 27.3 degree in an X-ray diffraction spectrum. This crystal type is generally called a Y-type or γ-type, and has high quantum efficiency of 90% or more in the intensity of an ordinary electrical field and is practically utilized for a super high sensitivity photoreceptor. The Y-type crystal shows a plurality of major absorption peaks in a long wavelength range, and has generally an absorption peak at a wavelength of around 800 nm and around 850 nm, and their intensity ratio can be changed according to manufacturing conditions. This crystal type is quasi-stable and is likely to be stabilized by exposing to heat, mechanical stress or contact with a solvent, thereby reducing the sensitivity. Also, this crystal type includes a water molecule in the crystal structure and is likely to change its property according to the humidity of the environment.

Also, U.S. Pat. No. 5,252,417 discloses a titanyl phthalocyanine crystal obtained from an amorphous titanyl phthalocyanine treated with monochlorobenzene and water. The titanyl phthalocyanine crystal has a major peak at about 27.3 degrees in an X-ray diffraction spectrum like a Y-type titanyl phthalocyanine crystal, but shows a different visible-infrared absorption spectrum pattern, and thus has a major absorption peak at a wavelength of around 790 nm and a minor absorption peak at around 710 nm.

U.S. Pat. No. 6,284,420 discloses a titanyl phthalocyanine crystal having a major absorption peak at a wavelength of around 790 nm in the visible-infrared absorption spectrum and a minor absorption peak having an intensity of 90% or more of the major absorption peak at about 700 nm.

Japanese Patent Laid-Open Gazette No. Hei 3-269061 discloses a crystal converting method in which noncrystalline or quasi-noncrystalline titanyl phthalocyanine is agitated using an alcoholic solvent, an aromatic solvent, or a mixed solvent of the alcoholic solvent or the aromatic solvent and water.

Japanese Patent Laid-Open Gazette No. Hei 10-073939 discloses a titanyl phthalocyanine crystal having a major absorption peak at a wavelength of around 780 nm in the visible-infrared absorption spectrum like the titanyl phthalocyanine crystal disclosed in U.S. Pat. No. 6,284,420. However, the intensity of its minor absorption peak significantly exceeds 80% of the major absorption peak.

U.S. Pat. No. 6,068,958 discloses a mixed crystal of titanyl phthalocyanine and copper phthalocyanine which has a major absorption peak at a wavelength of about 770 nm in the visible light-infrared absorption spectrum and a minor absorption peak at around 690 nm. However, the disclosed crystal is not a pure titanyl phthalocyanine crystal and the intensity of the minor absorption peak significantly exceeds ¾ of the major absorption peak.

### SUMMARY OF THE INVENTION

The present invention provides a titanyl phthalocyanine with a novel crystal type which has a high sensitivity equal to that of a Y-type titanyl phthalocyanine and overcomes the disadvantages of the Y-type crystal.

The present invention also provides a method of manufacturing the titanyl phthalocyanine with the novel crystal type.

The present invention further provides an electrophotographic photoreceptor using the titanyl phthalocyanine as a charge generating material.

The present invention also provides an electrophotographic image forming apparatus using the titanyl phthalocyanine as a charge generating material.

According to an aspect of the present invention, a titanyl phthalocyanine crystal is provided having a major absorption peak at a wavelength of 780 nm±10 nm in the visible-infrared absorption spectrum and a minor absorption peak having an intensity of ¾ or less of the major absorption peak at 700 nm±10 nm.

According to another aspect of the present invention, a method is provided for manufacturing a titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm by kneading a titanyl phthalocyanine source crystal having an absorption peak at a wavelength of 800 nm in the visible ray-infrared absorption spectrum with an alcoholic solvent.

According to another aspect of the present invention, an electrophotographic photoreceptor is provided comprising an electrically conductive substrate and a photosensitive layer formed on the electrically conductive substrate, wherein the photosensitive layer includes a titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm.

According to another aspect of the present invention, an electrophotographic image forming apparatus comprises: an electrophotographic photoreceptor comprising an electrically conductive substrate and a photosensitive layer formed on the electrically conductive substrate, wherein the photosensitive layer includes a titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm; a charging apparatus for charging the electrophotographic photoreceptor; an imagewise light irradiating device for irradiating imagewise light to the charged electrophotographic photoreceptor to form an electrostatic latent image on the electrophotographic photoreceptor; a developing unit for developing the electrostatic latent image with toner to form a toner image on the electrophotographic photoreceptor; and a transfer unit for transferring the toner image on an image receptor.

According to the present invention, the titanyl phthalocyanine crystal is obtained by kneading a titanyl phthalocyanine crystal having an absorption peak at a wavelength of around 800 nm in the visible-infrared absorption spectrum using an alcoholic solvent.

The titanyl phthalocyanine crystal according to the present invention has a high sensitivity equal to that of a Y-type titanyl phthalocyanine and overcomes the disadvantages of the Y-type crystal, thereby achieving good stability and a good electrophotographic characteristic.

These and other aspects of the invention will become apparent from the following detailed description of the invention and the annexed drawings which disclose various embodiments of the invention.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view of an image forming apparatus including an electrophotographic photoreceptor according to an embodiment of the present invention;

FIG. 2 illustrates a visible-infrared absorption spectrum of a titanyl phthalocyanine obtained from Synthesis Example 1 according to an embodiment of the present invention;

FIG. 3 illustrates an X-ray diffraction spectrum of a titanyl phthalocyanine obtained from Synthesis Example 1 according to an embodiment of the present invention;

FIG. 4 illustrates a visible-infrared absorption spectrum of a Y-type titanyl phthalocyanine used in Comparative Example 1;

FIG. 5 illustrates an X-ray diffraction spectrum of a Y-type titanyl phthalocyanine used in Comparative Example 1;

FIG. 6 illustrates a visible-infrared absorption spectrum of an α-type titanyl phthalocyanine used in Comparative Example 2; and

FIG. 7 illustrates an X-ray diffraction spectrum of an α-type titanyl phthalocyanine used in Comparative Example 2.

### DETAILED DESCRIPTION OF THE INVENTION

The present invention will now be described more fully with reference to the accompanying drawings, in which exemplary embodiments of the invention are shown. Throughout the drawings, the same drawing reference numerals will be understood to refer to the same elements, features and structures.

The visible-infrared absorption spectrum of the titanyl phthalocyanine crystal according to an embodiment of the present invention is remarkably different from that of a conventional well known titanyl phthalocyanine crystal. That is, except for a conventional β-type titanyl phthalocyanine crystal having low sensitivity, a conventional titanyl phthalocyanine crystal having a high sensitivity has a major absorption peak at a wavelength of 800 nm or greater. However, the titanyl phthalocyanine crystal according to an embodiment of the present invention has a major absorption peak at a wavelength of 780 nm±10 nm in the visible-infrared absorption spectrum and a minor absorption peak having an intensity of ¾ or less of the major absorption peak at a wavelength of 700 nm±10 nm. The lowest limit for the intensity of the minor absorption peak is not particularly defined.

The titanyl phthalocyanine crystal does not have a substantially independent absorption peak at a wavelength of 800 nm or greater.

The titanyl phthalocyanine crystal according to an embodiment of the present invention also differs remarkably from the conventional titanyl phthalocyanine crystal in the X-ray diffraction spectrum pattern. That is, the titanyl phthalocyanine crystal according to an embodiment of the present invention shows a distinct peak at a Bragg angle (2θ) of 9.2°, 14.5°, 18.1°, 24.1°, and 27.3° (all including an error of±0.2°). It is commonly observed that Y-type phthalocyanine has such a plurality of peaks, but titanyl phthalocyanine crystal according to an embodiment of the present invention does not have several peaks at 9.6°, 11.7°, 15.0° and so forth (all including an error of±0.2°) which is a characteristic of the X-ray diffraction spectrum pattern of the Y-type crystal. This indicates that the titanyl phthalocyanine crystal according to an embodiment of the present invention has a lattice constant similar to the Y-type titanyl phthalocyanine, but still has a difference in terms of lattice symmetry.

Also, the titanyl phthalocyanine crystal according to an embodiment of the present invention is similar to the titanyl phthalocyanine disclosed in U.S. Pat. No. 5,252,417 in that an absorption peak of 800 nm or more is not shown. However, the type of crystal can be distinguished by the location of the X-ray diffraction peak, the location of the visible-infrared absorption spectrum, and the difference of the intensity distribution. For example, The X-ray diffraction spectrum of the titanyl phthalocyanine crystal disclosed in U.S. Pat. No. 5,252,417 does not have a diffraction peak at a Bragg angle of 9.2°.

Also, the above titanyl phthalocyanine crystal according to an embodiment of the present invention having such a characteristic is distinguished from the crystals disclosed in Japanese Patent Laid-Open Gazette No. Hei 10-073939, U.S. Pat. No. 6,284,420, and U.S. Pat. No. 6,068,958, as described above.

The titanyl phthalocyanine crystal having the above characteristic according to an embodiment of the present invention is distinguished in the X-ray diffraction spectrum by the presence of peaks at 9.2°, 14.5°, and 18.1°, compared to the titanyl phthalocyanine crystal obtained by the crystal converting method disclosed in Japanese Patent Laid-Open Gazette No. Hei 3-0269061.

As described above, the titanyl phthalocyanine crystal according to an embodiment of the present invention is different in terms of the shape of the visible-infrared absorption spectrum and the X-ray diffraction spectrum, compared to the titanyl phthalocyanine crystals disclosed in the above mentioned publications.

Hereinafter, a method of manufacturing a titanyl phthalocyanine crystal will be described.

The titanyl phthalocyanine crystal according to an embodiment of the present invention uses a quasi-stable titanyl phthalocyanine crystal similar to an amorphous type (quasi α-type) or Y-type (γ-type) which is treated with acid paste as a source material. When the titanyl phthalocyanine is kneaded with an alcoholic solvent and, if necessary, with a binding resin, a titanyl phthalocyanine crystal with the above described properties can be obtained according to an embodiment of the present invention.

The acid-paste treatment is a well known method for purifying and/or changing the crystal from of pigments. For example, the process is disclosed in U.S. Pat. No. 5,106,536, the disclosure of which is hereby incorporated by reference in its entirety. Usually, it is carried out by a process comprising the steps of dissolving pigments into sulfuric acid completely, pouring the obtained acid solution into pure water for re-crystallizing and precipitating pigments, and separating the precipitated pigments from water.

Examples of the alcoholic solvent that can be used for the crystal conversion include a C₁₋₉ aliphatic lower alcohol. In particular, methanol, ethanol, propanol, butanol, and the like are appropriate because these alcohols can be easily handled. Also, these materials can be used alone or in a combination of two or more. These materials also can be used in a mixture with another solvent or with water in amounts that do not harm the effect of the present invention. For example, the alcoholic solvent and water can be used in the mixture ratio of 99/1 to 10/90, preferably 99/1 to 40/60.

The amount of solvent may be 1 to 100 times the weight amount of the titanyl phthalocyanine, preferably 2 to 10 times. The amount of the binding resin may be 0.1 to 100 times the weight amount of the titanyl phthalocyanine, preferably 0.2 to 5 times, more preferably 0.3 to 5 times the weight of the titanyl phthalocyanine.

Kneading can be performed using a kneading apparatus that can exert a high shear stress. Examples of the kneading apparatus include a kneader, a two-roll mill, a three-roll mill, an attritor, a ball-mill, a sand-mill, a Banbury mixer, a nanomizer, a microfluidizer, a stamp mill, a micronizer, a paint shaker, a high-speed agitator, an ultimizer, an ultrasonic mill, and the like. The apparatus may be used alone or in combination of two or more. Also, an appropriate amount of heat may be effective in crystal conversion during kneading. For example, kneading can be performed in a range from room temperature to about 200° C., preferably 50 to 150° C. The temperature should take into consideration the glass transition temperature of the binding resin. When a binding resin is used in combination, a solid dispersion obtained from the kneaded dispersion by crushing can be directly used to manufacture a composition (pigment) for forming a photosensitive layer, and thus a filtering step or a washing step using alcohol or water can be omitted as is performed in the conventional crystal conversion method.

The titanyl phthalocyanine obtained in the above kneading operation has a high sensitivity similar to the Y-type crystal and has a minute and uniform particle diameter. Thus, the titanyl phthalocyanine has good dispersion stability and the crystal state is stabilized. The titanyl phthalocyanine of the invention is more stable when exposed to heat and solvents than the Y-type crystal.

Hereinafter, an electrophotographic photoreceptor using the titanyl phthalocyanine crystal as a charge generating material according to an embodiment of the present invention will be described in detail.

The electrophotographic photoreceptor of the present embodiment includes an electrically conductive substrate and a photosensitive layer formed on the electrically conductive substrate, and includes the titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm. That is, the electrophotographic photoreceptor of the present embodiment includes a photosensitive layer formed on the electrically conductive substrate and the photosensitive layer includes the titanyl phthalocyanine crystal of an embodiment of the present invention.

The electrically conductive substrate can be made of any material having an electrical conductivity, such as a metal or a conductive polymer and may be in the form of a plate, a disk, a sheet, a belt, a drum, and the like. Examples of the metal include aluminum, vanadium, nickel, copper, zinc, palladium, indium, tin, platinum, stainless steel, and chromium. Examples of the conductive polymer include polyester resin, polycarbonate resin, polyamide resin, polyimide resin, and mixtures thereof. In other embodiments, a copolymer formed from monomers are used to synthesize resins having conductive materials dispersed therein, such as a conductive carbon, zinc oxide, indium oxide, or the like. A metal sheet or an organic polymer sheet on which metal is deposited or laminated may be also used as the electrically conductive substrate.

The photosensitive layer may be a laminated type where a charge generating layer and a charge transporting layer are separately formed or a single-layered type in which one layer has both a charge generating function and a charge transporting function.

The titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm, and having no substantial absorption peaks at a wavelength of 800 nm or greater, functions as a charge generating material. The crystal is also characterized by having a distinct peak at a Bragg angle (2θ) of 9.2°, 14.5°, 18.1°, 24.1°, and 27.3° (all including an error of±0.2°) in an X-ray diffraction spectrum.

If the photosensitive layer is a laminated type, the titanyl phthalocyanine crystal is included in the charge generating layer, and if the photosensitive layer is a single-layer type, the crystal is included in one photosensitive layer together with a charge generating layer.

Meanwhile, another known charge generating material can be mixed within an allowable amount not injuring the effect of the present invention. Examples of the known charge generating material that can be used in combination include organic materials such as phthalocyanine-based pigment other than the titanyl phthalocyanine crystal according to the present invention, azo-based pigment, quinone-based pigment, perylene-based pigment, indigo-based pigment, bisbenzoimidazole-based pigment, quinacridone-based pigment, azulenium-based dye, squarylium-based dye, pyryllium-based dye, triarylmethane-based dye, cyanine-based dye, etc. or inorganic materials such as amorphous silicon, amorphous selenium, trigonal selenium, tellurium, selenium-tellurium alloy, cadmium sulfide, antimony sulfide, zinc sulfide, etc.

In the case of a laminated type photosensitive layer, the charge generating material is dispersed in a solvent with a binder resin and deposited using a method such as dip coating, ring coating, roll coating, or spray coating to form a charge generating layer. The charge generating layer can also be formed using a vacuum deposition method, a sputtering method, or a chemical vapor deposition (CVD) method.

The thickness of the charge generating layer may be generally about 0.1-1 μm. If the thickness is less than 0.1 μm, the sensitivity is not sufficient, and if the thickness is more than 1 μm, the charging ability and the sensitivity decrease.

In the laminated type photosensitive layer, a charge transporting layer is formed on the charge generating layer. However, the charge generating layer may be formed on the charge transporting layer instead. In order to form a charge transporting layer, a solution in which a hole transporting material and a binding resin are dissolved in a solvent may be used for coating. The coating method may be dip coating, ring coating, roll coating, or spray coating like in the case of the charge generating layer. The thickness of the charge transporting layer may be generally 5 to 50 μm. If the thickness is less than 5 μm, the charging ability is not good, and if the thickness is greater than 50 μm, response speed and image quality decrease. The total thickness of the charge generating layer and the charge transporting layer are generally set within the range from 5 to 50 μm.

The amount of binding resin in the charge generating layer may be 5 to 350 parts by weight based on 100 parts by weight of the charge generating material, preferably 10 to 200 parts by weight. If the amount is less than 5 parts by weight, the dispersion of the titanyl phthalocyanine crystal in the present embodiment is not sufficient and thus it is difficult to obtain a uniform charge generating layer, and the adhesive force may also be degraded. If the amount is greater than 350 parts by weight, the charge potential is difficult to maintain and image quality decreases due to a reduced sensitivity.

In the charge transporting layer, the amount of the charge transporting material including a charge transporting material and/or a hole transporting material may be 10 to 60% by weight of the total weight of the charge transporting layer. If the amount of the charge transporting layer is less than 10% by weight, the charge transporting ability is not sufficient and thus the residual potential is likely to increase. If the amount is over 60% by weight, the amount of the resin in the charge transporting layer decreases and the mechanical intensity decreases.

In the case of a single-layer type photosensitive layer, a photosensitive layer is obtained by dispersing a charge generating material including the titanyl phthalocyanine crystal according to the present invention in a solvent together with a binding resin and a charge transporting material and coating. The thickness of the single-layer type photosensitive layer may be generally 5 to 50 μm.

Examples of the charge transporting material are a hole transporting material and an electron transporting material, however, the hole transporting material and the electron transporting material may be preferably used in combination, particularly, in the case of the single-layer type photosensitive layer. Since the single-layer type photosensitive layer uses a photosensitive layer in which a charge transporting material is dispersed with a charge generating material and a binding resin, charges are generated inside the photosensitive layer. Thus the photosensitive layer may preferably be capable of transporting both holes and electrons.

Examples of the hole transporting material include low molecular compounds such as nitrogen-containing cyclic compounds or fused-polycyclic compounds such as pyrene-based carbazole-based, hydrazone-based, oxazole-based, oxadiazole-based, pyrazolin-based, arylamine-based, arylmethane-based, benzidine-based, thiazol-based, stilbene-based, or butadiene-based compounds. Also, high molecular weight compounds or polysilane compounds having a functional group of the above compounds on a main chain or a side chain can be used. Examples of the high molecular weight compounds include poly-N-vinyl carbazole, halogenated poly-N-vinyl carbazole, polyvinyl pyrene, polyvinyl anthracene, polyvinyl acridine, pyrene formaldehyde resin, ethyl carbazole formaldehyde resin, and triphenylmethane polymer.

Examples of the electron transporting material include electron withdrawing low molecular compounds such as benzo quinone-based, tetracyanoethylene-based, tetracyanoquinodimethane-based, fluorenone-based, xanthone-based, phenanthraquinone, phthalic anhydride-based, diphenoquinone-based, stilbene quinone-based, naphthalene-based, thiopyran-based compounds, etc. Also, electron transporting polymer compounds having the above listed electron withdrawing low molecular compounds on a main chain or a side chain or electron transporting pigments such as titanium oxide, zinc oxide, cadmium sulfide, and so forth can also be used.

The electron transporting material or the hole transporting material used for the electrophotographic photoreceptor is not limited hereto. Also, the material may be used singularly or in a combination of two or more.

The binding resin used to form a coating solution for forming a photosensitive layer may be a polymer which can form an electrically insulating film. Examples of the polymer include, but are not limited to, polycarbonate, polyester, methacrylic resin, acrylic resin, polyvinyl chloride, polyvinylidene chloride, polystyrene, polyvinylacetate, styrene-butadiene copolymer, vinylidene chloride-acrylonitrile copolymer, vinyl chloride-vinyl acetate copolymer, vinyl chloride-vinyl acetate-maleic anhydride copolymer, silicone resin, silicone-alkyd resin, phenol-formaldehyde resin, styrene-alkyd resin, poly-N-vinyl carbazole, polyvinyl butyral, polyvinyl formal, polysulfone, casein, gelatin, polyvinyl alcohol, ethyl cellulose, phenol resin, polyamide, carboxy methyl cellulose, vinylidene chloride-based polymer latex, polyurethane, etc. The binding resin may be used singularly or in a combination of two or more.

The amount of charge transporting material including an electron transporting material and/or a hole transporting material in the single-layer type photosensitive layer may be 10 to 60% by weight based on the total weight of the photosensitive layer. If the amount is less than 10% by weight, the charge transporting ability is not sufficient, and thus the sensitivity is not sufficient and the residual current is likely to increase. If the amount is over 60% by weight, the amount of resin in the photosensitive layer decreases, and thus, the mechanical intensity is likely to decrease.

Regardless of whether a photosensitive layer is a laminated type or single-layer type, the electrophotographic photoreceptor may include additives such as a plasticizer, a surface modifier, a dispersion stabilizer, an antioxidant, an optical stabilizer, etc. in the photosensitive layer together with the binding resin.

Examples of the plasticizer include, but are not limited to, biphenyl, chlorinated biphenyl, terphenyl, dibutyl phthalate, diethylene glycol phthalate, dioctyl phthalate, triphenyl phosphate, methyl naphthalene, benzophenone, chlorinated paraffin, polypropylene, polystyrene, and a fluorinated hydrocarbon.

Examples of the surface modifier are silicon oil and fluorine resin, etc.

Also, the photosensitive layer may include an antioxidant or optical stabilizer in order to improve resistance to chemical attack or stability to harmful light. Examples of the compounds that can be used for this purpose include, but are not limited to, chromanol derivatives such as tocopherol and its etherified compound or esterified compound, a poly aryl alkane compound, and a hydroquinone derivative and its mono- and dietherified compound, a benzophenone derivative, a benzotriazole derivative, an sulfided ether compound, a phenylenediamine derivative, phosphonic acid ester, phosphite ester, a phenolic compound, a sterically hindered phenolic compound, a straight-chain amine compound, a cyclic amine compound, and a sterically hindered amine compound.

An intermediate layer may be further interposed between the electrically conductive substrate and the photosensitive layer in order to improve adhesive force or prevent charge injection from the electrically conductive substrate. Examples of the intermediate layer include, but are not limited to, an anodic oxide layer of aluminum (alumite layer), a resin dispersion layer of metal oxide powder like titanium oxide, tin oxide, etc., and resin layers like poly vinyl alcohol, casein, ethyl cellulose, gelatin, phenol resin, polyamide, etc. The thickness of the intermediate layer may be preferably in the range of 0.05 to 5 μm.

The electrophotographic photoreceptor according to the present invention may further include a surface protection layer when necessary.

When the photosensitive layer is formed using a dip coating method, the above described amount of charge generating material and/or charge transporting material with a binding resin is dissolved or dispersed in a solvent in order to be used as a composition for forming the photosensitive layer. The solvent which dissolves the binder resin may vary depending on the type of binding resin. Examples of the organic solvent include alcohols such as methanol, ethanol, n-propanol, isopropanol, 1-butanol, 2-butanol, 1-methoxy-2-propanol, etc.; ketones such as acetone, methyl ethyl ketone, cyclohexanone, methyl isopropyl ketone, methyl isobutyl ketone, 4-methoxy-4-methyl-2-penthanone, etc.; amides such as N,N-dimethylformamide, N,N-dimethyl acetamide, etc.; ethers such as tetrahydrofurane, dioxane, methyl cellosolve, etc.; esters such as methyl acetate, ethyl acetate, isopropyl acetate, t-butyl acetate, etc.; sulfoxides such as dimethyl sulfoxide, etc.; aliphatic halogenized hydrocarbons such as 1,2-dichloroethane, 1,1,2-tricholoroethane, 1,1,1-tricholoroethane, trichloroethylene, tetracholoroethane, dichloromethane, methylene chloride, chloroform, carbon tetrachloride, tricholoroethane, etc.; aromatics such as benzene, toluene, ethyl benzene, xylene, monochlorobenzene, dichlorobenzene, etc.; and amines such as butyl amine, diethylamine, ethylene diamine, isopropanol, amine triethanolamine, triethylene, diamine, etc. Whether a laminated type or single-layer type, the solvent preferably does not have an adverse effect on an adjacent layer.

The electrophotographic photoreceptor according to an embodiment of the present invention can be integrated in electrophotographic image forming apparatuses such as laser printers, photocopiers, facsimile machines, LED printers, etc.

The electrophotographic image forming apparatus includes an electrophotographic photoreceptor including an electrically conductive substrate and a photosensitive layer formed on the electrically conductive layer, wherein the photosensitive layer includes a titanyl phthalocyanine crystal according to an embodiment of the present invention having a major absorption peak at a wavelength of 780 nm±10 nm in the visible-infrared absorption spectrum and a minor absorption peak having an intensity of ¾ or less of the major absorption peak at 700 nm±10 nm, a charging apparatus for charging the electrophotographic photoreceptor, an imagewise light irradiating device that irradiates imagewise light to the charged electrophotographic photoreceptor in order to form an electrostatic latent image on the electrophotographic photoreceptor, a developing unit for developing the electrostatic latent image with toner in order to form a toner image on the electrophotographic photoreceptor, and a transfer unit for transferring the toner image onto an image receptor.

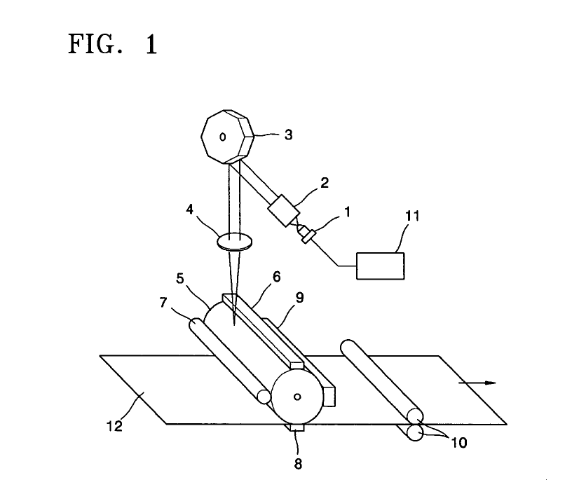

FIG. 1 schematically illustrates an electrophotographic image forming apparatus according to an embodiment of the present invention. Referring to FIG. 1, reference numeral 1 refers to a semiconductor laser. Laser light that is signal-modulated by a control circuit 11 according to image information, after being radiated is collimated by an optical correction system 2 and performs scanning while being reflected by a polygonal rotatory mirror 3. The laser light is focused on a surface of an electrophotographic photoreceptor 5 by a scanning lens 4 and exposes the surface according to the image information. Since the electrophotographic photoreceptor is already charged by a charging apparatus 6, an electrostatic latent image is formed by the exposure, and then becomes visible by a developing apparatus 7. The visible image is transferred to an image receptor 12 such as paper by a transferring apparatus 8, and is fixed in a fixing apparatus 10 and provided as a print result. The electrophotographic photoreceptor can be used repeatedly by removing coloring agent that remains on the surface thereof by a cleaning apparatus 9. The electrophotographic photoreceptor here is drawn in the form of a drum; however, as described above, it may also be in the form of a sheet or a belt.

Hereinafter, the present invention will be described in more detail with reference to the following examples. However, these examples are given for the purpose of illustration and are not intended to limit the scope of the invention. The term “parts” in the examples refer to “parts by weight” unless indicated otherwise.

### FABRICATION EXAMPLE 1

2 parts of Y-type titanyl phthalocyanine which is synthesized according to the method disclosed in U.S. Pat. No. 4,898,799 and 1 part of polyvinyl butyral resin (“S-LEC BM-1”, available from Sekisui Chemical Co., Ltd.) are mixed with 5 parts of isopropyl alcohol and kneaded using a two-roll mill (R2-type available from Kodaira Seisakusho Co., Ltd.) The obtained dispersion is dried in an oven at 100° C. for 1 hour and crushed to obtain a solid dispersion with a chip shape.

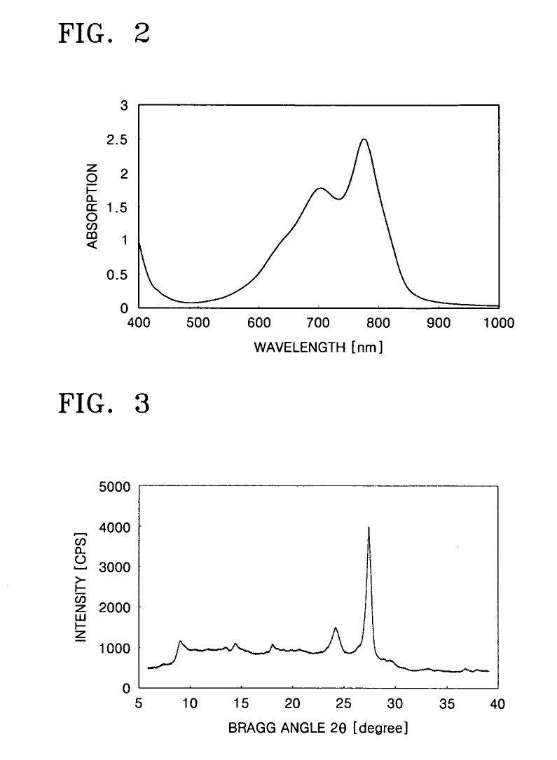

FIGS. 2 and 3 respectively illustrate a visible-infrared absorption spectrum and an X-ray diffraction spectrum of Fabrication Example 1.

Referring to FIG. 2, the titanyl phthalocyanine crystal has a major absorption peak at a wavelength of around 780 nm in the visible ray-infrared absorption spectrum, and a minor absorption peak which has about 70% of the intensity of the major absorption peak at around 700 nm. Also, the titanyl phthalocyanine crystal does not have an absorption peak at a wavelength of 800 nm or greater.

Referring to the X-ray diffraction spectrum in FIG. 3, the titanyl phthalocyanine crystal has a distinct diffraction peak at a Bragg angle (2θ) of 9.2°, 14.5°, 18.1°, 24.1°, and 27.3°.

### FABRICATION EXAMPLE 2

A pasty mixture in which an amorphous titanyl phthalocyanine (“Fastgen Blue 8310” available from Dainippon Ink & Chemicals) obtained by acid paste treatment, and n-butanol were mixed with equal amounts, was kneaded using a pressurized kneader (“ND-0.5”, available from Naniwa Kikai Kogyo Co., Ltd.) while raising the temperature to 80° C. for 20 minutes. Then the kneaded pigment was dried using a vacuum drier to remove the remaining solvent. The absorption spectrum and the X-ray diffraction spectrum of the pigment exhibited the same properties as those of the resultant material obtained from Fabrication Example 1.

### EXAMPLE 1

A coating composition obtained by dissolving 4 parts of the solid dispersion obtained from Fabrication Example 1 with 96 parts of ethanol was coated using a ring coating method on an anodized aluminum drum having a diameter of 30 mm and dried to form a charge generating layer to a thickness of about 0.4 μm. Then a solution in which 60 parts of polycarbonate resin Z (“lupilon Z-200”, available from Mitsubishi Gas Chemical) and 40 parts of aryl amine-based hole transporting material represented by Formula 1 below were dissolved in 300 parts of chloroform was coated thereon and was dried at 100° C. for 1 hour to form a charge transporting layer to a thickness of 20 μm. Thus a laminated type electrophotographic photoreceptor was obtained.

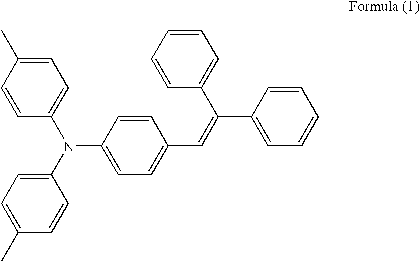

### COMPARATIVE EXAMPLE 1

2 parts of a Y-type titanyl phthalocyanine and 1 part of a polyvinyl butyral resin (“LEC BM-1”, available from Sekisui Chemical Co., Ltd.) were dispersed with 17 parts of ethanol using a sand mill for 1 hour. The obtained dispersion solution was agitated while adding ethanol dropwise to obtain a coating composition with 4% solid content. This composition was coated on an anodized aluminum drum as in Embodiment 1 using a ring coating method and dried to form a charge generating layer to a thickness of about 0.4 μm.

And, a charge transporting layer was formed in the same manner as the charge generating layer in Example 1 and a laminated type electrophotographic photoreceptor was obtained.

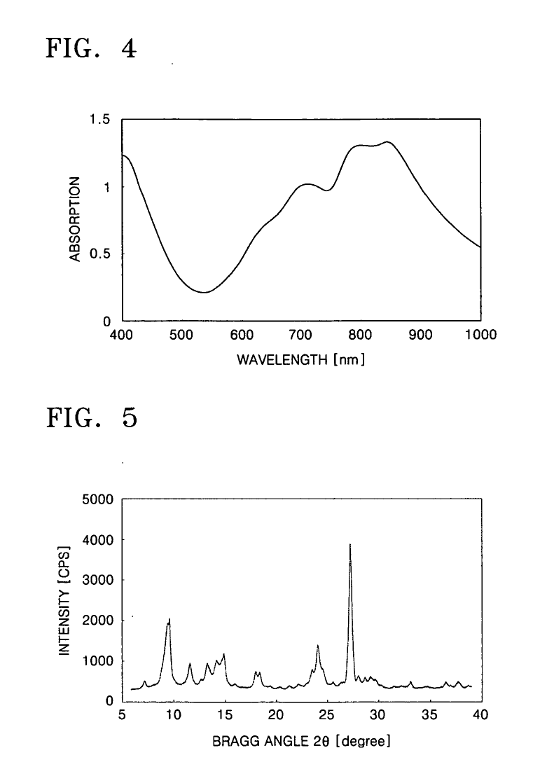

FIGS. 4 and 5 show the visible-infrared absorption spectrum and the X-ray diffraction spectrum of the Y-type titanyl phthalocyanine used in Comparative Example 1.

Comparing FIGS. 4 and 5 with FIGS. 2 and 3, respectively, a remarkable difference can be seen in the visible ray-infrared absorption pattern and the X-ray diffraction pattern.

### EXAMPLE 2

An electrophotographic photoreceptor was obtained in the same manner as in Comparative Example 1, except that a pigment obtained from Fabrication Example 2 was used instead of the Y-type titanyl phthalocyanine.

COMPARATIVE EXAMPLE 2

An electrophotographic photoreceptor was obtained in the same manner as in Comparative Example 1, except that an α-type titanyl phthalocyanine was used instead of the Y-type titanyl phthalocyanine.

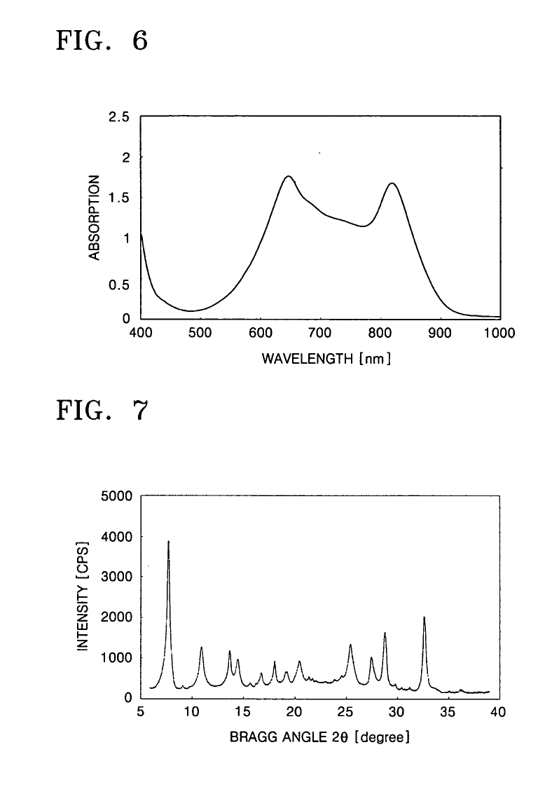

FIGS. 6 and 7 show the visible-infrared absorption spectrum and the X-ray diffraction spectrum of the α-type titanyl phthalocyanine used in Comparative Example 2.

Comparing FIGS. 6 and 7 with FIGS. 2 and 3, respectively, a remarkable difference can be seen in the visible ray-infrared absorption pattern and the X-ray diffraction pattern.

### MEASUREMENT OF ELECTROPHOTOGRAPHIC PROPERTIES

The characteristics of the electrophotographic properties of the photoreceptors respectively obtained from Examples and Comparative Examples were measured using an electrostatic characteristic evaluator (“PDT-2000” available from QEA) at 23° C. at a humidity of 50% (N/N) and at a temperature of 10° C. and at a humidity of 20% (L/L) as follows.

Each photoreceptor was charged with a corona voltage of −7.5 kV and a relative speed of the charging apparatus to the photoreceptor being 100 mm/sec, and then immediately a monochromatic light having a wavelength of 780 nm in the range of exposure energy of 0 to 5 mJ/m² was irradiated to the photoreceptors. The surface potential after exposure was recorded to measure the relationship between the energy and the surface potential. Here, the surface potential when no light was radiated was referred to as V₀ [V], and the potential after exposure of 5 mJ/m² was referred to as Vᵢ [V]. Also, exposure energy required for V₀ to decrease by ½ is denoted as E_{1/2}[mJ/m²]. The measurements are listed in Table 1.

TABLE 1

| | Environment | V₀ (V) | Vᵢ (V) | E_{1/2} (mJ/m²) |
|---|---|---|---|---|
| Example 1 | N/N | −748 | −36 | 1.05 |
| | L/L | −723 | −47 | 1.17 |
| Comparative | N/N | −724 | −35 | 1.03 |
| Example 1 | L/L | −695 | −62 | 1.25 |
| Example 2 | N/N | −734 | −38 | 1.06 |
| | L/L | −718 | −49 | 1.18 |
| Comparative | N/N | −722 | −94 | 2.43 |
| Example 2 | L/L | −708 | −115 | 2.76 |

Referring to Table 1, the photoreceptors of Examples 1 and 2 have good sensitivity and stability in any environments. Meanwhile, the photoreceptor in Comparative Example 1 using a Y-type titanyl phthalocyanine shows high sensitivity similar to that of the photoreceptors in Examples 1 and 2 in an N/N environment. In an L/L environment, the sensitivity decreases and residual current is large. In the case of the photoreceptor in Comparative Example 2 using an α-type titanyl phthalocyanine, the sensitivity is half that of the photoreceptor of Examples 1 and 2, and residual current is very large.

As seen from the results, the photoreceptor of the present invention has good sensitivity and residual current property, and moreover, has good stability to varying environments. Accordingly, high quality images can be stably obtained using the electrophotographic photoreceptor according to the present invention.

As described above, the titanyl phthalocyanine crystal of the present invention has high sensitivity similar to that of the Y-type crystal and the particle diameter thereof is minute and uniform due to the kneading process. Accordingly, with good dispersion stability and being in the stabilized crystal state, the titanyl phthalocyanine crystal is much more stable to heat or solvents. The photoreceptor using the titanyl phthalocyanine of the present invention as a charge generating material has good sensitivity and a good residual current property, and in addition, good stability. Accordingly, high quality images can be stably obtained using the electrophotographic photoreceptor of the present invention.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm.

2. The titanyl phthalocyanine crystal of claim 1, wherein the titanyl phthalocyanine crystal does not have an absorption peak at a wavelength of 800 nm or greater.

3. The titanyl phthalocyanine crystal of claim 1, wherein the titanyl phthalocyanine crystal has distinct peaks at a Bragg angle (2θ) of 9.2°, 14.5°, 18.1°, 24.1°, and 27.3° (±0.2°).

4. A method of manufacturing a titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm, said method comprising kneading a titanyl phthalocyanine source material crystal having an absorption peak at a wavelength of 800 nm in the visible ray-infrared absorption spectrum with an alcoholic solvent.

5. The method of claim 4, wherein a binder resin is further included in the kneading step.

6. The method of claim 4, wherein the titanyl phthalocyanine source material crystal is an amorphous type titanyl phthalocyanine or a Y-type (γ-type) titanyl phthalocyanine treated with acid paste.

7. The method of claim 5, wherein the amount of the alcoholic solvent is 1 to 100 times the weight of the titanyl phthalocyanine source material crystal, and the amount of the binder resin is 0.1 to 100 times the weight of the titanyl phthalocyanine source material crystal.

8. The method of claim 4, wherein the titanyl phthalocyanine crystal does not have an absorption peak at a wavelength of 800 nm or greater.

9. An electrophotographic photoreceptor comprising an electrically conductive substrate and a photosensitive layer formed on the electrically conductive substrate, wherein the photosensitive layer includes a titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm.

10. The electrophotographic photoreceptor of claim 9, wherein the photosensitive layer is a single-layer type photosensitive layer comprising a charge generating function and a charge transporting function at the same time.

11. The electrophotographic photoreceptor of claim 9, wherein the photosensitive layer is a laminated type comprising a charge generating layer and a charge transporting layer, and the titanyl phthalocyanine crystal is included in the charge generating layer.

12. The electrophotographic photoreceptor of claim 9, wherein the titanyl phthalocyanine crystal does not have an absorption peak at a wavelength of 800 nm or greater.

13. The electrophotographic photoreceptor of claim 9, wherein the titanyl phthalocyanine crystal has distinct peaks at a Bragg angle (2θ) of 9.2°, 14.5°, 18.1°, 24.1°, and 27.3° (all including an error of±0.2°).

14. The electrophotographic photoreceptor of claim 9, wherein the titanyl phthalocyanine crystal is obtained by kneading a titanyl phthalocyanine crystal having an absorption peak at a wavelength of 800 nm in the visible-infrared absorption spectrum using an alcoholic solvent.

15. An electrophotographic image forming apparatus comprising:
an electrophotographic photoreceptor comprising an electrically conductive substrate and a photosensitive layer formed on the electrically conductive substrate, wherein the photosensitive layer includes a titanyl phthalocyanine crystal having a major absorption peak at a wavelength of 780 nm±10 nm in the visible-infrared absorption spectrum and a minor absorption peak having an intensity of ¾ or less of the major absorption peak at 700 nm±10 nm;
a charging apparatus for charging the electrophotographic photoreceptor;
an imagewise light irradiating device for irradiating imagewise light to the charged electrophotographic photoreceptor in order to form an electrostatic latent image on the electrophotographic photoreceptor;
a developing unit for developing the electrostatic latent image with toner in order to form a toner image on the electrophotographic photoreceptor; and
a transfer unit for transferring the toner image on an image receptor.

16. The electrophotographic image forming apparatus of claim 15, wherein the photosensitive layer is a single-layer type photosensitive layer comprising a charge generating function and a charge transporting function at the same time.

17. The electrophotographic image forming apparatus of claim 15, wherein the photosensitive layer is a laminated type comprising a charge generating layer and a charge transporting layer, and the titanyl phthalocyanine crystal is included in the charge generating layer.

18. The electrophotographic photoreceptor of claim 15, wherein the titanyl phthalocyanine crystal does not have an absorption peak at a wavelength of 800 nm or greater.

19. The electrophotographic photoreceptor of claim 15, wherein the titanyl phthalocyanine crystal has distinct peaks at a Bragg angle (2θ) of 9.2°, 14.5°, 18.1°, 24.1°, and 27.3° (all including an error of±0.2°).
